(19) United States
(12) Patent Application Publication
(10) Pub. No.: US 2025/0339793 A1
(43) Pub. Date: Nov. 6, 2025

(54) FOAM CONTROL AGENTS FORMED FROM POLYOL ESTER OF FATTY ACIDS (PEFAS)

(71) Applicant: Ruby Bio Inc., San Carlos, CA (US)

(72) Inventors: Pavan Kumar Reddy Kambam, Sunnyvale, CA (US); Denis Kembero Ontumi, Dublin, CA (US); Charles Scott Silver, Oakland, CA (US)

(21) Appl. No.: 18/869,009

(22) PCT Filed: May 26, 2023
(86) PCT No.: PCT/US2023/067582

§ 371 Date: Nov. 25, 2024

Related U.S. Application Data
(60) Provisional application No. 63/365,404, filed on May 26, 2022.
(60) Provisional application No. 63/365,405, filed on May 26, 2022.
(60) Provisional application No. 63/365,406, filed on May 26, 2022.

(51) Int. Cl. B01D 19/04; A61K 8/37; A61K 8/46; A61K 8/55; A61Q 19/00; C05G 3/00; C09D 7/63; C09J 11/06; C09K 3/10

(52) U.S. Cl. CPC B01D 19/0404; A61K 8/375; A61K 8/463; A61K 8/55; A61Q 19/00; B01D 19/0418; B01D 19/0422; C05G 3/00; C09D 7/63; C09J 11/06; C09K 3/10; C09K 2200/0441

(57) ABSTRACT

A foam control agent or formulation including at least one polyol lipid having a polyol ester of fatty acid (PEFA) compound.

### REFERENCE TO RELATED APPLICATIONS

This application claims priority to U.S. Provisional Patent Application Ser. No. 63/365,404, entitled Compounds, Compositions, and Methods of Use Thereof, filed May 26, 2022; U.S. Provisional Patent Application Ser. No. 63/365,405, entitled Polyol Ester of Fatty Acids (PEFAs) as Antifoam, filed May 26, 2022; and U.S. Provisional Patent Application Ser. No. 63/365,406, entitled Polyol Ester of Fatty Acids (PEFAs) as Emulsifiers, filed May 26, 2022; and hereby incorporates the provisional applications by reference herein in their entireties.

### TECHNICAL FIELD

The present disclosure relates to foam control agents and formulations comprising polyol esters of fatty acids (PEFAs), and methods for reducing, preventing, or controlling foam.

### BACKGROUND

Foam is often produced as an unwanted consequence in the manufacture of various substances, such as manufacturing foodstuffs, particularly in processes involving significant shear forces near air-liquid interfaces, such as those involving aeration, pumping or agitation. Foam control agents are added to manufacturing processes to reduce foam generation.

Many conventional surfactants (both petroleum and bio-based) used in a broad variety of agricultural, nutritional, cosmetic, veterinary, therapeutic, and industrial applications can cause significant foaming. Further, petroleum-based surfactants can exhibit poor performance with regard to sustainability, bio-accumulation, eco-toxicity and/or biodegradability.

Therefore, there is a need for developing renewable, low-toxicity, biodegradable foam control agents that reduce or eliminate foaming for various applications.

### BRIEF DESCRIPTION OF THE DRAWINGS

FIG. 1 is a graph depicting foam generation height for various compositions containing foam-generating compounds.

### DETAILED DESCRIPTION

The details of various embodiments of the invention are set forth in the description below. Other features, objects, and advantages of the invention will be apparent from the description and the drawings, and from the claims.

### Definitions

As used herein, the term “foam control agent” refers to foam inhibitors that prevent the generation of foam (e.g., antifoamers), and/or reduce foam after it is formed (e.g., defoamers).

As used herein, the term “foam control formulation” refers to a formulation comprising a foam control agent that prevents the formation of foam (e.g., antifoamers), and/or reduce or suppress foam after it is formed (e.g., defoamers).

As used herein, the term “foam” refers to a dispersion of a gas (usually air) in a liquid or solid.

As used herein, a “foodstuff” refers to material that is edible or drinkable, or a material that can be processed into an edible or drinkable material.

As used herein, weight percent (wt %), percent by weight, % by weight, and the like are synonyms that refer to the concentration of a substance as the weight of that substance divided by the weight of the composition and multiplied by 100.

The term “about”, preceding a FIGURE encompasses plus or minus 10%, or less, of the value of said FIGURE. It is to be understood that the value to which the term “about” refers is itself also specifically, and preferably, disclosed.

The term “alkyl” refers to a straight or branched alkyl group. Exemplary alkyl groups are methyl, ethyl, propyl, isopropyl, butyl, isobutyl, and tert-butyl.

The term “halogen” means F, Cl, Br, or I.

As used herein, the term “salt or acid thereof” refers to any salts or acids of PEFA compounds. Exemplary salts and acids of PEFA compounds are suitable for use in contact with the tissues of humans and lower animals without undue toxicity, irritation, allergic response and the like, and are commensurate with a reasonable benefit/risk ratio. Salts can include pharmaceutically or biologically acceptable salts. Likewise, acids can include pharmaceutically or biologically acceptable acids. Pharmaceutically or biologically acceptable salts and acids are well known in the art. For example, S. M. Berge et al., describe pharmaceutically or biologically acceptable salts in detail in J. Pharmaceutical Sciences, 1977, 66, 1-19, incorporated herein by reference. Pharmaceutically or biologically acceptable salts of the compounds of this disclosure can include those derived from suitable inorganic and organic acids and bases, while pharmaceutically or biologically acceptable acids of the compounds of this disclosure can include suitable inorganic and organic acids. Examples of pharmaceutically or biologically acceptable inorganic acids can include hydrochloric acid, hydrobromic acid, phosphoric acid, sulfuric acid, nitric acid and perchloric acid or with organic acids such as acetic acid, oxalic acid, maleic acid, tartaric acid, citric acid, succinic acid, propionic acid or malonic acid or by using other methods used in the art such as ion exchange. Examples of pharmaceutically or biologically acceptable acid addition salts are salts of an amino group formed with such examples of inorganic acids. Another example of a pharmaceutically or biologically acceptable acid is a carboxylic acid, where the carboxylated version of the PEFA compound includes a carboxyl group. Other pharmaceutically or biologically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C1-4alkyl)4 salts. Representative alkali or alkaline earth metal salts include sodium, lithium, potassium, calcium, magnesium, and the like. Further pharmaceutically or biologically acceptable salts include, when appropriate, ammonium, quaternary ammonium, and amine cations formed using counterions such as halide, hydroxide, carboxylate, sulfate, phosphate, nitrate, lower alkyl sulfonate and aryl sulfonate.

The present disclosure includes both the (R) and (S) configuration at each stereo center, even in cases where drawn as defined or drawn undefined. Additionally, the present disclosure includes racemic compositions of compounds disclosed herein. The present disclosure includes present disclosure includes scalemic compositions of compounds disclosed herein. The present disclosure includes present disclosure includes enantioenriched compositions of compounds disclosed herein.

Polyol lipid biosurfactants as described herein can include polyol esters of fatty acids (PEFAs).

Polyol lipids can be isolated and/or purified from a yeast culture according to methods known in the art. Since the polyol lipids are extracellularly secreted from yeast cells, and yeast cell cultures do not contain a hydrophobic carbon source, the one or more polyol lipids can be isolated and/or purified from the yeast cell culture without cell lysis or extraction requiring an organic solvent.

Polyol esters of fatty acids are amphiphilic molecules comprising a sugar alcohol, e.g., a D-mannitol and/or a D-arabitol, esterified to the carboxyl end of a 3-hydroxy fatty acyl moiety, which may or may not be acetylated. The non-esterified hydroxy groups of the sugar alcohol may or may not be acetylated as well. In some embodiments, the one or more polyol lipids produced are a mixture of similar compounds containing (R)-3-hydroxy fatty acyl moieties with varying chain lengths, in the range of about 8 to 24 carbons, preferably in the range between 12 to 20 carbons. In some embodiments, the (R)-3-hydroxy fatty acyl moieties can present varying degrees of unsaturation in the range of about 0 to 6, e.g., in the range between 2 to 5. In some embodiments, the non-esterified hydroxy groups of the sugar alcohol can be esterified to acetyl groups. In some embodiments the sugar alcohol can be fully acetylated. In some embodiments, the sugar alcohol can be non-acetylated. In some embodiments, acetylations can range between these two states.

In some embodiments, a polyol lipid disclosed herein is an acetylated C12:0-3-hydroxy fatty acid esterified to D-arabitol with 3 acetylations. In some embodiments, a polyol lipid disclosed herein is an acetylated C14:0-3-hydroxy fatty acid esterified to D-mannitol with 4 acetylations. In some embodiments, a polyol lipid disclosed herein is an acetylated C16:0-3-hydroxy fatty acid esterified to D-mannitol with 2 acetylations. In some embodiments, a polyol lipid disclosed herein is an acetylated C16:0-3-hydroxy fatty acid esterified to D-arabitol with 2 acetylations. In some embodiments, a polyol lipid disclosed herein is an acetylated C14:0-3-hydroxy fatty acid esterified to D-arabitol with 4 acetylations. In some embodiments, a polyol lipid disclosed herein is an acetylated C14:0-3-hydroxy fatty acid esterified to D-mannitol with 5 acetylations. In some embodiments, a polyol lipid disclosed herein is an acetylated C16:0-3-hydroxy fatty acid esterified to D-mannitol with 3 acetylations. In some embodiments, a polyol lipid disclosed herein is an acetylated C16:0-3-hydroxy fatty acid esterified to D-arabitol with 3 acetylations. In some embodiments, a polyol lipid disclosed herein is an acetylated C16:0-3-hydroxy fatty acid esterified to D-mannitol with 4 acetylations. In some embodiments, a polyol lipid disclosed herein is an acetylated C18:0-3-hydroxy fatty acid esterified to D-mannitol with 2 acetylations. In some embodiments, a polyol lipid disclosed herein is an acetylated C16:0-3-hydroxy fatty acid esterified to D-mannitol with 5 acetylations. In some embodiments, a polyol lipid disclosed herein is an acetylated C16:0-3-hydroxy fatty acid esterified to D-mannitol with 4 acetylations. In some embodiments, the polyol lipid is an acetylated C18:0-3-hydroxy fatty acid esterified to D-mannitol with 2 acetylations. In some embodiments, a polyol lipid disclosed herein is an acetylated C16:0-3-hydroxy fatty acid esterified to D-mannitol with 5 acetylations. In some embodiments, a polyol lipid disclosed herein is an acetylated C16:0-3-hydroxy fatty acid esterified to D-arabitol with 4 acetylations. In some embodiments, a polyol lipid disclosed herein is an acetylated C18:0-3-hydroxy fatty acid esterified to D-mannitol with 3 acetylations. In some embodiments, a polyol lipid disclosed herein is an acetylated C18:0-3-hydroxy fatty acid esterified to D-mannitol with 4 acetylations. In some embodiments, a polyol lipid disclosed herein is an acetylated C18:0-3-hydroxy fatty acid esterified to D-arabitol with 3 acetylations. In some embodiments, a polyol lipid disclosed herein is an acetylated C18:0-3-hydroxy fatty acid esterified to D-mannitol with 5 acetylations. In some embodiments, a polyol lipid disclosed herein is an acetylated C18:0-3-hydroxy fatty acid esterified to D-arabitol with 4 acetylations. In some embodiments, a polyol lipid disclosed herein is an acetylated C20:0-3-hydroxy fatty acid esterified to D-mannitol with 3 acetylations. In some embodiments, a polyol lipid disclosed herein is an acetylated C20:0-3-hydroxy fatty acid esterified to D-mannitol with 4 acetylations.

### Exemplary Compounds

In some embodiments, a polyol lipid is a PEFA compound represented by a compound selected from the group consisting of:

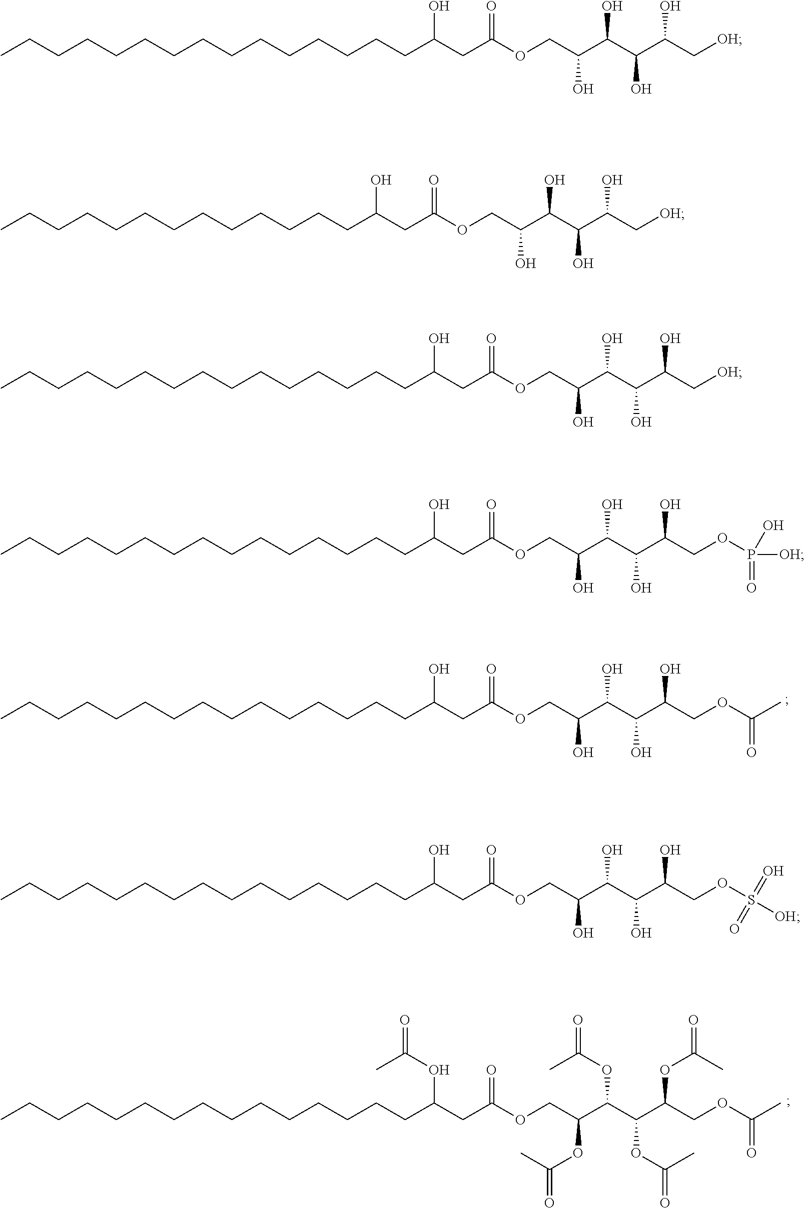

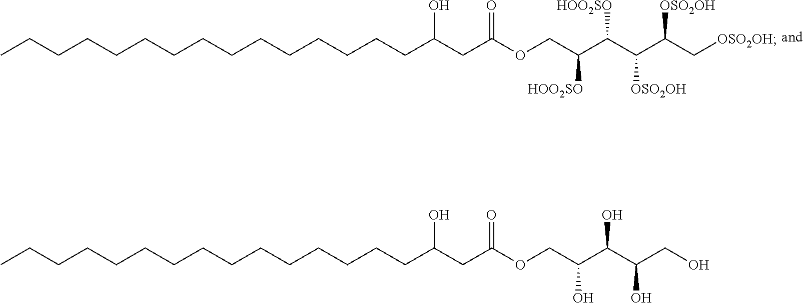

or salt or acid thereof.

### Additional Exemplary Compounds

In some embodiments, a polyol lipid is a PEFA compound represented by Formula (I) or (II):

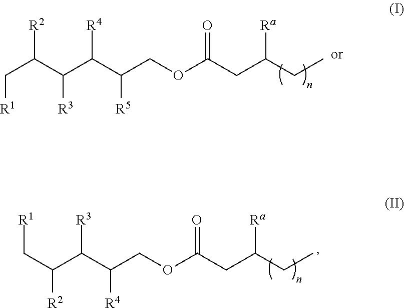

or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁴ is selected from the group consisting of —H, —C(O)R, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{y}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁵ is selected from the group consisting of —H, —C(O)R, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{y}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{y}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and
each p is independently selected from 1-10.

In some embodiments, a polyol lipid is a PEFA compound of Formula (I) or (II):

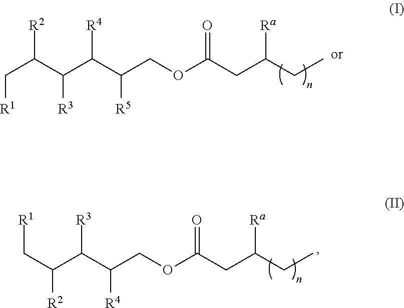

or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{y}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R⁵ is selected from the group consisting of —H, —C(O)R^{x}, —R^{y}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{y}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{y}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, —(O)(CⱼHₖOₘ);
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and
each p is independently selected from 1-10,
wherein
   when n is 12 or 14 and R¹ is —OH or —(O)Ac, then R^{a} is not —(O)Ac or —(O)Me;
   when n is 12 and R^{a} is —OH, then R² is not —OH or;
   when n is 12 or 14, R^{a} is —OH, R² is OH, R³ is OH, and R⁴ is OH, then R¹ is (O)R^{x} or —(O)(C₂H₅O)ₚR^{y};
   when n is 12 or 14, R¹ is —(O)S(O₂)OH, R² is —(O)S(O₂)OH, R³ is —(O)S(O₂)OH, R⁴ is —(O)S(O₂)OH, and R⁵ is —(O)S(O₂)OH, then R^{a} is —(O)C(O)R^{x}, R^{x}, —(O)C(O)OR^{x}, —(O)C(O)N(H)R^{x}, —(O)(C₂H₅O)ₚR^{y}, —(O)P(O)(OH)₂, or —(O)S(O₂)OH.

In some embodiments, a compound of Formula (I) or (II), wherein
R¹ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R² is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R³ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R⁴ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R⁵ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R^{a} is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-11; and
each p is independently selected from 1-10.

In some embodiments, a polyol lipid is a PEFA compound represented by Formula (I-a) or (II-a):

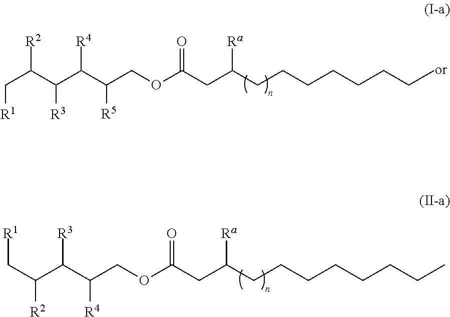

or salt or acid thereof,
wherein
n is 6-12.

### R¹

In some embodiments, R¹ is selected from the group consisting of —H, —C(O)R^{x}, —R^{x}, —C(O)OR, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{y}, —(O)R^{x}, —(O)C(O)OR, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ). In some embodiments, R¹ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R¹ is —H. In some embodiments, R¹ is —P(O)(OH)₂, or —S(O₂)OH. In some embodiments, R¹ is —C(O)R^{x}. In some embodiments, R¹ is —Ac. In some embodiments, R¹ is —R^{x}. In some embodiments, R¹ is —C(O)OR^{y}. In some embodiments, R¹ is —C(O)N(H)R^{y}. In some embodiments, R¹ is —(C₂H₅O)ₚR^{y}. In some embodiments, R¹ is —P(O)(OH)₂. In some embodiments, R¹ is —S(O₂)OH. In some embodiments, R¹ is —(CⱼHₖOₘ).

### R²

In some embodiments, R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ). In some embodiments, R² is selected from the group consisting of —C(O)R^{x}, —R^{x}, —C(O)OR^{y}, —C(O)N(H)R^{y}, —(C₂H₅O)ₚR, —P(O)(OH)₂, and —S(O₂)OH. In some embodiments, R² is —H. In some embodiments, R² is —P(O)(OH)₂, or —S(O₂)OH. In some embodiments, R² is —C(O)R^{x}. In some embodiments, R² is —Ac. In some embodiments, R² is —R^{x}. In some embodiments, R² is —C(O)OR^{y}. In some embodiments, R² is —C(O)N(H)R^{y}. In some embodiments, R² is —(C₂H₅O)ₚR^{y}. In some embodiments, R² is —P(O)(OH)₂. In some embodiments, R² is —S(O₂)OH. In some embodiments, R² is —(CⱼHₖOₘ).

### R³

In some embodiments, R³ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R³ is —H. In some embodiments, R³ is —P(O)(OH)₂, or —S(O₂)OH. In some embodiments, R³ is —C(O)R^{x}. In some embodiments, R³ is —Ac. In some embodiments, R³ is —R^{x}. In some embodiments, R³ is —C(O)OR^{y}. In some embodiments, R³ is —C(O)N(H)R^{y}. In some embodiments, R³ is —(C₂H₅O)ₚR^{y}. In some embodiments, R³ is —P(O)(OH)₂. In some embodiments, R³ is —S(O₂)OH. In some embodiments, R³ is —(CⱼHₖOₘ). In some embodiments, R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ).

### R⁴

In some embodiments, R⁴ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R⁴ is —H. In some embodiments, R⁴ is —P(O)(OH)₂, or —S(O₂)OH. In some embodiments, R⁴ is —C(O)R^{x}. In some embodiments, R⁴ is —Ac. In some embodiments, R⁴ is —R^{x}. In some embodiments, R⁴ is —C(O)OR^{y}. In some embodiments, R⁴ is —C(O)N(H)R^{y}. In some embodiments, R⁴ is —(C₂H₅O)ₚR^{y}. In some embodiments, R⁴ is —P(O)(OH)₂. In some embodiments, R⁴ is —S(O₂)OH. In some embodiments, R⁴ is —(CⱼHₖOₘ). In some embodiments, R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ).

### R⁵

In some embodiments, R⁵ is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R⁵ is —H. In some embodiments, R⁵ is —P(O)(OH)₂, or —S(O₂)OH. In some embodiments, R⁵ is —C(O)R^{x}. In some embodiments, R⁵ is —Ac. In some embodiments, R⁵ is —R^{x}. In some embodiments, R⁵ is —C(O)OR^{y}. In some embodiments, R⁵ is —C(O)N(H)R^{y}. In some embodiments, R⁵ is —(C₂H₅O)ₚR^{y}. In some embodiments, R⁵ is —P(O)(OH)₂. In some embodiments, R⁵ is —S(O₂)OH. In some embodiments, R⁵ is —(CⱼHₖOₘ). In some embodiments, R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ).

### R^{a}

In some embodiments, R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ). In some embodiments, R^{a} is selected from the group consisting of —C(O)R^{x}, —R^{x}, —C(O)OR^{y}, —C(O)N(H)R^{y}, —(C₂H₅O)ₚR^{y}, —P(O)(OH)₂, and —S(O₂)OH. In some embodiments, R^{a} is —H. In some embodiments, R^{a} is —P(O)(OH)₂, or —S(O₂)OH. In some embodiments, R^{a} is —C(O)R^{x}. In some embodiments, R^{a} is —Ac. In some embodiments, R^{a} is —R^{x}. In some embodiments, R^{a} is —C(O)OR^{y}. In some embodiments, R^{a} is —C(O)N(H)R^{y}. In some embodiments, R^{a} is —(C₂H₅O)ₚR^{y}. In some embodiments, R^{a} is —P(O)(OH)₂. In some embodiments, R^{a} is —S(O₂)OH. In some embodiments, R^{a} is —(CⱼHₖOₘ).

### R^{x}

In some embodiments, each R^{x} is independently C₁-C₃ alkyl, wherein R^{x} is optionally substituted with 1-7 instances of halogen. In some embodiments, R^{x} is methyl, wherein R^{x} is optionally substituted with 1-7 instances of halogen. In some embodiments, R^{x} is ethyl, wherein R^{x} is optionally substituted with 1-7 instances of halogen. In some embodiments, R^{x} is n-propyl, wherein R^{x} is optionally substituted with 1-7 instances of halogen. In some embodiments, R^{x} is i-propyl, wherein R^{x} is optionally substituted with 1-7 instances of halogen. In some embodiments, R^{x} is methyl. In some embodiments, R^{x} is ethyl. In some embodiments, R^{x} is n-propyl. In some embodiments, R^{x} is i-propyl.

### R^{y}

In some embodiments, each R^{y} is independently —H or C₁-C₃ alkyl, wherein R^{y} is optionally substituted with 1-7 instances of halogen. In some embodiments, R^{y} is —H. In some embodiments, each R^{y} is independently C₁-C₃ alkyl, wherein R^{y} is optionally substituted with 1-7 instances of halogen. In some embodiments, R^{y} is methyl, wherein R^{y} is optionally substituted with 1-7 instances of halogen. In some embodiments, R^{y} is ethyl, wherein R^{y} is optionally substituted with 1-7 instances of halogen. In some embodiments, R^{y} is n-propyl, wherein R^{y} is optionally substituted with 1-7 instances of halogen. In some embodiments, R^{y} is i-propyl, wherein R^{y} is optionally substituted with 1-7 instances of halogen. In some embodiments, R^{y} is methyl. In some embodiments, R^{y} is ethyl. In some embodiments, R^{y} is n-propyl. In some embodiments, R^{y} is i-propyl.

In some embodiments, a polyol lipid is a PEFA compound represented by:

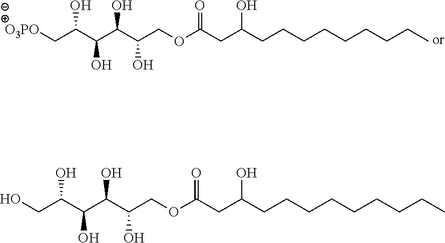

or salt or acid thereof.

### Preparation of Compounds

Below are examples of specific embodiments regarding the invention described by the present disclosure. The examples are offered for illustrative purposes only and are not intended to limit the scope of the present invention in any way. The groups referenced in the examples below, e.g., OR¹, OR², etc., refer to the corresponding groups described herein, e.g., R¹, R², etc., and can be replaced with any of the groups provided in the respective corresponding group, i.e., without the “O.” Efforts have been made to ensure accuracy with respect to numbers used (e.g., amounts, temperatures, etc.), but some experimental error and deviation should, of course, be allowed for.

In some embodiments, compounds of the present disclosure can be prepared as outlined in Scheme 1 or 2:

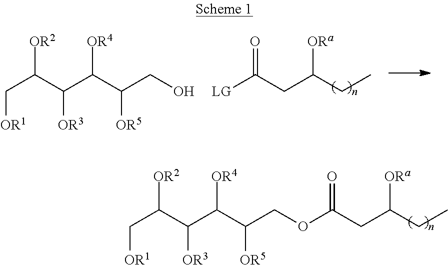

wherein LG is a leaving group.

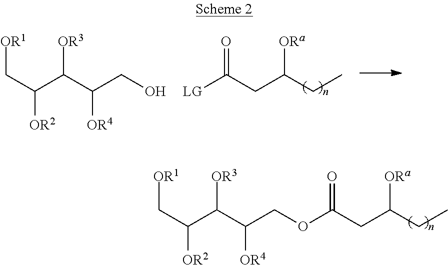

wherein LG is a leaving group.

In some embodiments, compounds of the present disclosure can be prepared as outlined in Scheme 3 or 4:

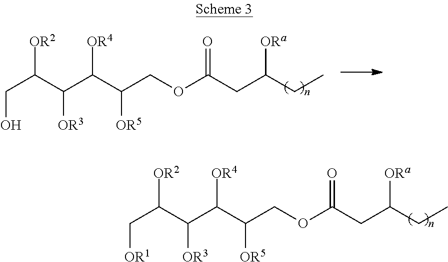

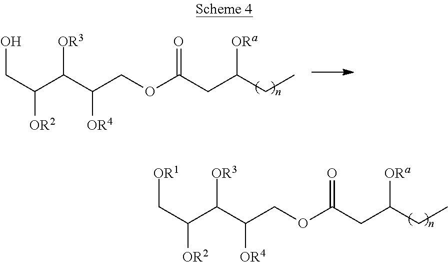

In some embodiments, compounds of the present disclosure can be prepared as outlined in Scheme 5 or 6:

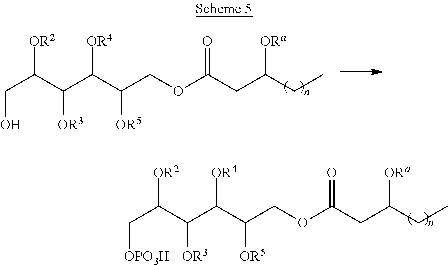

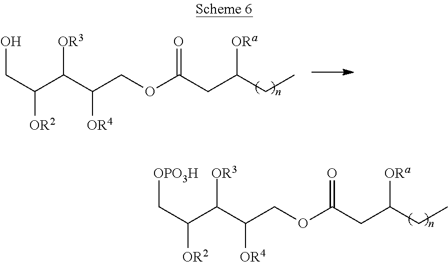

Synthesis/isolation of starting materials for the compounds described herein can be found in WO2018148465 and WO2017184884, each of which are incorporated in their entirety.

A non-limiting example of preparation of Hydroxy-dodecanoic acid 2,3,4,5-tetrahydroxy-6-phosphonooxy-hexyl ester:

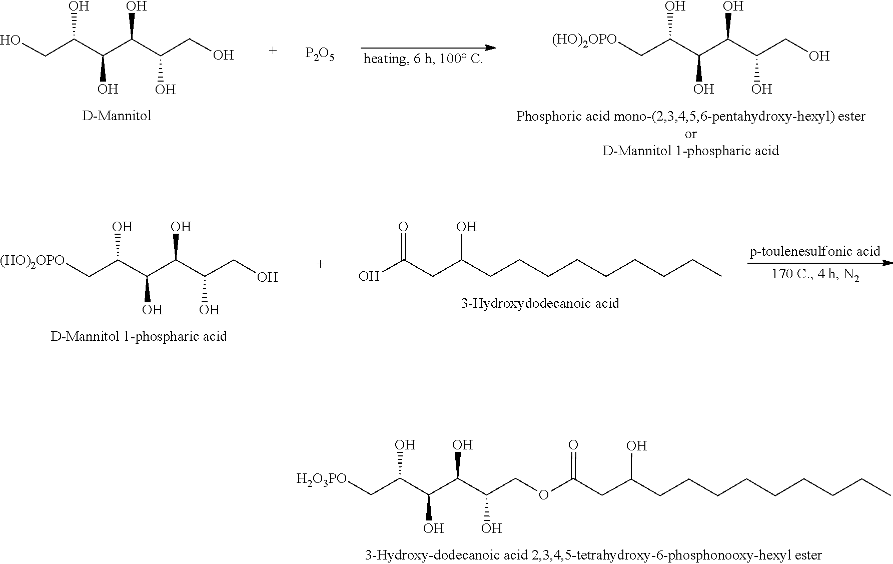

A non-limiting example of preparation of D-Mannitol 1-phosphoric acid: D-mannitol and P2O5 are heated neat at 100° C. for 6 hours. As an example, D-Mannitol 1-phosphoric acid can be prepared as outlined in J.-G. Nam et al. Materials Chemistry and Physics 116 (2009) 46-51. D-Mannitol 1-phosphoric acid and 3-hydroxydodecanoic acid are heated in p-toluene sulfonic acid at 170° C. for 4 h at N2 as outlined in Wenyuan Han et al. Polymers 11, (2019), 1031.

### Foam Control

Foam is an impediment to the manufacture of various compositions and products, and disrupts the production and process of making such compositions and products. Reducing, suppressing or eliminating foam can be of substantial assistance in the production of many personal care products to render the production more efficient and effective.

To address the foaming problem, conventional foam control agents or formulations have been utilized. But these conventional foam control agents may not sufficiently reduce the amount of foam, or may exhibit poor performance with regard to sustainability, bio-accumulation, eco-toxicity and/or biodegradability. Foam control agents and formulations disclosed herein provide advantages over other conventional agents and formulations in that they are renewable, have low-toxicity, and/or have good biodegradablility while still providing foam control.

The foam control agents or formulations described herein can be introduced at a single point in a processing operation or can be introduced at one or more points along the manufacturing process. Foam control agents or formulations disclosed herein can be used in any one or more processing steps and formulated in compositions suitable for each type of processing method.

In some embodiments, a foam control agent is inert so as to not react with the other components of a product or system in which it is introduced. In some embodiments, a foam control agent reacts minimally with the components of a product or system in which it is introduced. In some embodiments, a foam control agent has no adverse effect on a product or system. In some embodiments, a foam control agent has minimal adverse effect on the product or system. In some embodiments, a foam control agent is present in an end product of a processing procedure. In some embodiments, a foam control agent is physiologically safe or has a safety profile that is biologically acceptable. In some embodiments, a foam control agent is biodegradable and/or environmentally safe or environmentally acceptable.

A foam control agent can include at least one polyol lipid. In some embodiments, at least one polyol lipid is a polyol ester of fatty acid (PEFA) compound. The PEFA compound can include one of the PEFA compounds described herein, including those represented by Formula (I), (II), (I-a) or (II-a). In some embodiments, a foam control agent can comprise two or more polyol lipids. In some embodiments, the two or more polyol lipids are polyol esters of fatty acids (PEFAs) compounds. In some embodiments, the two or more polyol lipids are selected from PEFA compounds described herein, including those represented by Formula (I), (II), (I-a), or (II-a).

In some embodiments, a foam control agent can have pentitol polyol esters of fatty acids and hexitol polyol esters of fatty acids. In some embodiments, the C16 and C18 fatty acid components of the pentitol and hexitol polyol esters can comprise about 20% to about 99% of the PEFA; and in some embodiments from about 30% to about 95% of the PEFA; in some embodiments from about 40% to about 90% of the PEFA. In some embodiments, the C16 and C18 fatty acid components of the pentitol and hexitol polyol esters can comprise about 50% or greater of the PEFA; in some embodiment, about 60% or greater of the PEFA; and in some embodiments, about 70% or greater of the PEFA; and in some embodiments, about 80% or greater of the PEFA. In some embodiments, the ratio of pentitol to hexitol polyol esters in the surfactant is about 5:1 or greater; in some embodiment, the ratio is about 8:1 or greater; in some embodiments, the ratio is about 10:1 or greater; in some embodiments, the ratio is about 15:1 or greater; in some embodiments is about 20:1 or greater; in some embodiments, the ratio is about 30:1 or greater; in some embodiments, the ratio is about 40:1 or greater; in some embodiments is about 50:1 or greater.

In some embodiments, a foam control formulation disclosed herein further comprises a solvent, a carrier, an additive, a stabilizing agent, a surfactant, an emulsifier, or a combination thereof.

In some embodiments, a foam control formulation disclosed herein can further include a solvent. Such solvents can be selected for dissolving or dispersing the at least one polyol lipid. Suitable solvents include any of a variety of solvents that solubilize but do not significantly degrade the at least one polyol lipid.

Non-limiting examples of solvents include hydrocarbons (both aromatic and aliphatic), oxygenated solvents (alcohols, ketones, aldehydes, ethers, glycol ethers, esters, and glycol ether esters), polyalkylene oxide, capped polyalkylene oxide, and combinations thereof. Suitable polyalkylene oxides include polyethylene glycol, polypropylene glycol, polybutylene glycol, mixtures thereof, or the like. Suitable capped polyalkylene oxides include mono-alkyl and di-alkyl ethers of the respective polyalkylene oxides, such as mono- and di-methyl ethers of polyalkylene glycol, mono- and di-ethyl ethers of polyalkylene glycol, mono- and di-propyl ethers of polyalkylene glycol, mono- and di-butyl ethers of polyalkylene glycol, mixtures thereof, or the like. Suitable capped polyalkylene oxides include methyl polyethylene glycol (e.g., the monomethyl ether of polyethylene glycol), dimethyl polyethylene glycol (e.g., the dimethyl ether of polyethylene glycol), mixtures thereof, or the like.

Suitable solvent solubilizers include glycol ethers. Suitable glycol ethers include diethylene glycol n-butyl ether, diethylene glycol n-propyl ether, diethylene glycol ethyl ether, diethylene glycol methyl ether, diethylene glycol t-butyl ether, dipropylene glycol n-butyl ether, dipropylene glycol methyl ether, dipropylene glycol ethyl ether, dipropylene glycol propyl ether, dipropylene glycol tert-butyl ether, ethylene glycol butyl ether, ethylene glycol propyl ether, ethylene glycol ethyl ether, ethylene glycol methyl ether, ethylene glycol methyl ether acetate, propylene glycol n-butyl ether, propylene glycol ethyl ether, propylene glycol methyl ether, propylene glycol n-propyl ether, tripropylene glycol methyl ether and tripropylene glycol n-butyl ether, ethylene glycol phenyl ether (commercially available as DOWANOL EPH™ from Dow Chemical Co.), propylene glycol phenyl ether (commercially available as DOWANOL PPH™ from Dow Chemical Co.), and the like, or mixtures thereof. Additional suitable commercially available glycol ethers (all of which are available from Union Carbide Corp.) include Butoxyethyl PROPASOL™, Butyl CARBITOL™ acetate, Butyl CARBITOL™, Butyl CELLOSOLVE™ acetate, Butyl CELLOSOLVE™, Butyl DIPROPASOL™, Butyl PROPASOL™, CARBITOL™ PM-600, CARBITOL™ Low Gravity, CELLOSOLVE™ acetate, CELLOSOLVE™, Ester EEP™, FILMER IBT™, Hexyl CARBITOL™, Hexyl CELLOSOLVE™, Methyl CARBITOL™, Methyl CELLOSOLVE™ acetate, Methyl CELLOSOLVE™, Methyl DIPROPASOL™, Methyl PROPASOL™ acetate, Methyl PROPASOL™, Propyl CARBITOL™, Propyl CELLOSOLVE™, Propyl DIPROPASOL™ and Propyl PROPASOL™.

In some embodiments, a foam control formulation disclosed herein further comprises a carrier. A carrier can provide a medium which dissolves, suspends, or carries the other components of a foam control composition. For example, the carrier can provide a medium for solubilization, suspension, or production of polyol lipid and for forming an equilibrium mixture. The carrier can also function to deliver antimicrobial components of a foam control composition on an object. To this end, the carrier can contain any component or components that can facilitate such delivery and serve additional functions for the foam control composition.

In certain embodiments, the carrier includes primarily water which can promote solubility and work as a medium for reaction and equilibrium. Non-limiting examples of carriers can include one or more of hydrocarbon oils, water, fatty alcohols and esters, and organic solvents, such as simple alkyl alcohols, e.g., ethanol, isopropanol, n-propanol, and the like.

In certain embodiments, the carrier makes up a large portion of foam control formulation. The carrier concentration and type will depend upon the nature of the formulation, the environmental storage, and method of application including concentration of the polyol lipid, among other factors.

In certain embodiments, a foam control formulation includes about 0 to about 98 wt % carrier, about 0.001 to about 99.99 wt % carrier, about 0.2 to about 60 wt % carrier, about 1 to about 98 wt % carrier, about 5 to about 99.99 wt % carrier, about 5 to about 97 wt % carrier, about 5 to about 90 wt % carrier, about 5 to about 70 wt % carrier, about 5 to about 20 wt % carrier, about 10 to about 90 wt % carrier, about 10 to about 80 wt % carrier, about 10 to about 50 wt % carrier, about 10 to about 20 wt % carrier, about 15 to about 70 wt % carrier, about 15 to about 80 wt % carrier, about 20 to about 70 wt % carrier, about 20 to about 50 wt % carrier, about 20 to about 40 wt % carrier, about 20 to about 30 wt % carrier, about 30 to about 75 wt % carrier, about 30 to about 70 wt % carrier, about 40 to about 99.99 wt % carrier, about 40 to about 90 wt % carrier, or about 60 to about 70 wt % carrier.

In some embodiments, a foam control formulation can further include one or more additives. Non-limiting examples of additives include ethylene oxide/propylene oxide block copolymers, butylene oxide/propylene oxide block copolymers, ethylene oxide/butylene oxide block copolymers, waxes, or silicone-based materials.

In some embodiments, a foam control formulation can further include a stabilizing agent. The shelf-life of a foam control formulation as disclosed herein can be improved by including a stabilizing agent and/or a preservative to prevent bacterial spoilage. Non-limiting examples of stabilizing agents include oleic acid, hexylene glycol, fatty alcohols, naphthalene sulfonates, butyl alcohol, and formaldehyde.

In some embodiments, a surfactant or secondary emulsifier is selected to be added to the water-in-oil emulsion or oil-in-water emulsion so as to improve the wettability of the emulsion. For example, the improved wettability is useful when such emulsions are applied on a foodstuff, or when such emulsions are formed with at least one polyol lipid. In some embodiments, such surfactants or secondary emulsifiers may be anionic, cationic, nonionic or amphoterics and combination thereof. Non-limiting examples of anionic surfactants or secondary emulsifiers include alkali metal, ammonium and amine soaps; the fatty acid part of such soaps contains preferably at least 16 carbon atoms. Other non-limiting examples of anionic surfactants or secondary emulsifiers include alkali metal salts of alkvl-aryl sulfonic acids, sodium dialkyl sulfosuccinate, sulfated or sulfonated oils, e.g., sulfated castor oil; sulfonated tallow, and alkali salts of short chain petroleum sulfonic acids. Non-limiting examples of cationic surfactants or secondary emulsifiers include salts of long chain primary, secondary or tertiary amines, such as oleylamide acetate, cetylamine acetate, di-dodecylamine lactate, the acetate of aminoethyl-aminoethyl stearamide, dilauroyl triethylene tetramine diacetate, 1-aminoethyl-2-heptadecenyl imidazoline acetate; and quaternary salts, such as cetylpyridinium bromide, hexadecyl ethyl morpholinium chloride, and diethyl di-dodecyl ammonium chloride. Non-limiting examples of nonionic surfactants or secondary emulsifiers include condensation products of higher fatty alcohols with ethylene oxide, such as the reaction product of oleyl alcohol with 10 ethylene oxide units; condensation products of alkylphenols with ethylene oxide, such as the reaction product of isoctylphenol with 12 ethylene oxide units; condensation products of higher fatty acid amides with 5, or more, ethylene oxide units; polyethylene glycol esters of long chain fatty acids, such as tetraethylene glycol monopalmitate, hexaethyleneglycol monolaurate, nonaethyleneglycol monostearate, nonaethyleneglycol dioleate, tridecaethyleneglycol monoarachidate, tricosaethyleneglycol monobehenate, tricosaethyleneglycol dibehenate, polyhydric alcohol partial higher fatty acid esters such as sorbitan tristearate, ethylene oxide condensation products of polyhydric alcohol partial higher fatty acid esters, and their inner anhydrides (mannitol-anhydride, called Mannitan, and sorbitol-anhydride, called Sorbitan), such as glycerol monopalmitate reacted with 10 molecules of ethylene oxide, pentaerythritol monooleate reacted with 12 molecules of ethylene oxide, sorbitan monostearate reacted with 10-15 molecules of ethylene oxide, mannitan monopalmitate reacted with 10-15 molecules of ethylene oxide; long chain polyglycols in which one hydroxyl group is esterified with a higher fatty acid and other hydroxyl group is etherified with a low molecular alcohol, such as methoxypolyethylene glycol 550 monostearate (550 meaning the average molecular weight of the polyglycol ether). Non-limiting examples of amphoteric surfactants include alkybetaine, alkyldimethylamine N-oxide, alkylamidopropylamine N-oxide, alkylamidopropylbetaine, cocamidopropyl betaine, cocoamphoacetate and cocoamphodiacetate, phosphatidylserine, phosphatidylethanolamine, phosphatidylcholine, sphingomyelins, lauryldimethylamine oxide, and myristamine oxide. In some embodiments, a combination of two or more of these surfactants may be used; e.g., a cationic may be blended with a nonionic or an anionic with a nonionic.

One of ordinary skill in the art will appreciate that the individual components of a foam control formulation may change depending on the physical and chemical qualities needed for the foam control in a given process and/or application to which the formulation will be applied. For example, the dispersibility of foam control formulation in water can be adjusted as necessary to obtain the desired performance.

Hydrophilic-lipophilic balance (HLB) of a surfactant is a measure of the degree to which it is hydrophilic or lipophilic. In some embodiments, a foam control agent disclosed herein has a HLB of 0 to 12. In some embodiments, a foam control agent disclosed herein has a HLB of less than 10. In some embodiments, a foam control agent disclosed herein is lipid-soluble (water-insoluble). In some embodiments, a foam control agent disclosed herein has a HLB of 1 to 3. In some embodiments, a foam control formulation disclosed herein has a HLB of 0 to 12. In some embodiments, a foam control formulation disclosed herein has a HLB of less than 10. In some embodiments, a foam control formulation disclosed herein is lipid-soluble (water-insoluble). In some embodiments, a foam control formulation disclosed herein has a HLB of 1 to 3. Measurement of HLB is determined according to methods known in the art (e.g., calculation according to Kothencz et al. (2017) Studia Universitatis Babes-bolyai Chemia 62: 451-458).

The foam control agents described herein can also serve as a co-foam control agents with either another PEFA-based foam control agent or a non-PEFA-based foam control agent.

The foam control agents and formulations described herein are particularly relevant to are variety of applications, including food and beverage, cosmetic and dermatologic, home care, personal care, industrial cleaning, auto care, oil fracking, pharmaceutical, agriculture, paint, adhesives, inks, flavors and fragrances.

A method of reducing or preventing the generation of foam when manufacturing a composition for a product (e.g., food or beverage) can include having the foam control agent or formulation introduced before, during, or after said foam is generated during the manufacturing process. This timing of when to introduce a foam control agent or formulation as described herein would also apply to the manufacture of products in various applications, including cosmetic and dermatologic, home care, personal care, industrial cleaning, auto care, oil fracking, pharmaceutical, agriculture, paint, adhesives, inks, flavors and fragrances.

In certain embodiment, the amount of foam reduced or suppressed by a foam control agent or formulation could be about 10% or more; in certain embodiments about 20% or more; in certain embodiments about 30% or more; in certain embodiments about 40% or more; in certain embodiments about 50% or more; in certain embodiments about 60% or more; in certain embodiments about 70% or more; in certain embodiments about 80% or more; in certain embodiments about 85% or more; in certain embodiments about 90% or more; in certain embodiments about 95% or more; and in certain embodiments about 100%.

### Food/Beverage

During food and beverage processing and manufacturing foodstuffs, undesirable foam formation can occur at various processing stages during the processing of a foodstuff. The foam is caused by the presence of surface-active substances such as proteins, fatty acids and sugars when aeration (generated for example by mechanical agitation, mixing, washing, extraction, stirring, sparging, etc.) occurs during processing.

In some embodiments, a foodstuff includes a foam control agent and/or a foam control formulation as described herein and has improved properties as compared conventional agents. Nonlimiting examples of improved properties can include, for example, decreased or increased foam generation, decreased or increased foam decay, decreased or increased in the density of the liquor/filtrate as the foam control agent of foam control formulation is introduced (e.g., measure of the change in density of a filtrate is a direct measurement of the change in entrained air), decreased or increased surface foam. In some embodiments, a foam control agent disclosed herein is added to a foodstuff in sufficient quantity to achieve the level of foam control necessary for a process. It is recognized that different food processing techniques result in varying levels of foam generation, and as such, require varying amounts of foam control agent to achieve the desired result.

In some embodiments, the amount of foam control agent added to a food stuff is measured as a percentage of the combined weight of the foam control agent and the foodstuff (total weight of the food composition). In some embodiments, the quantity of the foam control agent is from about 0.001 to 0.5 percent, by weight, of the total weight of the food composition, or about 0.01 to 5 percent, by weight, of the total weight of the food composition. In some embodiments, the quantity of the foam control agent is from about 0.1 to 1 percent, by weight, of the total weight of the food composition. In some embodiments, the quantity of the foam control agent is from about 0.01 to 2 percent, by weight, of the total weight of the food composition, or about 0.01 to about 1 percent, by weight, of the total weight of the food composition.

Non-limiting examples of foodstuffs can include, for example, edible plants, vegetables, fruits, and grains, and derivatives of edible plants, vegetables, fruits, and grains that are formed when these foods are subject to processing using methods known in the art.

Nonlimiting examples of processes using foodstuffs where foam control is desirable can include, for example during industrial processing of sugar beet (such as leading to formation of sugar, syrups, and juices) foam formation can occur in processing equipment during washing, cutting, diffusing, carbonizing, and evaporation steps; during industrial processing of potatoes, foam formation can occur in processing equipment during washing, cleaning, polishing, and cutting; and industrial fermentation processes, including fermentation.

Additional non-limiting examples of processes can include, for example, food processing such as cleaning agents for fresh and frozen fruits, vegetables, meats and processed foods, food processing such as rheologic modifiers in food applications including doughs, pastas, and food processing such as antiadherents.

In some embodiments, a food composition or a beverage disclosed herein does not comprise a petroleum-based surfactant.

### Cosmetic/Dermatologic

Due to skin-friendly properties, the polyol lipids disclosed herein can be used in cosmetics and dermatologic compositions.

In some embodiments, a cosmetic or dermatologic composition includes a foam control agent and/or a foam control formulation as described herein and has improved properties as compared conventional agents. Nonlimiting examples of improved properties can include, for example, decreased or increased foam generation, decreased or increased foam decay, decreased or increased in the density of the liquor/filtrate as the foam control agent of foam control formulation is introduced (e.g., measure of the change in density of a filtrate is a direct measurement of the change in entrained air), decreased or increased surface foam.

In some embodiments, the amount of foam control agent added to a cosmetic is measured as a percentage of the combined weight of the foam control agent and the cosmetic (total weight of the cosmetic or dermatological composition). In some embodiments, the quantity of the foam control agent is from about 0.001 to 0.5 percent, by weight, of the total weight of the cosmetic or dermatological composition, or about 0.01 to 5 percent, by weight, of the total weight of the cosmetic or dermatological composition. In some embodiments, the quantity of the foam control agent is from about 0.1 to 1 percent, by weight, of the total weight of the cosmetic or dermatological composition. In some embodiments, the quantity of the foam control agent is from about 0.01 to 2 percent, by weight, of the total weight of the cosmetic or dermatological composition, or about 0.01 to about 1 percent, by weight, of the total weight of the cosmetic or dermatological composition.

Non-limiting examples of cosmetic and dermatological compositions can include, for example, low foam hand washes, and solid skin care compositions.

Additional non-limiting examples of cosmetic and dermatological compositions can include, for example, creams, foams, mousses, balms, ointments, personal care formulations such as shampoos, body washes, conditioners, soaps, creams, skin treatments, and moisturizing agents.

Additional non-limiting examples of cosmetic and dermatological compositions can include, for example, an anti-perspirant, a skin cleansing agent, a skin conditioner, a skin cosmetic composition product, a make-up composition product, an oil-based cosmetic composition product, a skin care cosmetic composition product, a hair cleansing agent, an external use preparation for hair or a hair cosmetic composition product.

In some embodiments, a cosmetic or dermatological composition disclosed herein, wherein the composition does not comprise a petroleum-based surfactant.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

To determine the impact a PEFA compound can have on foaming, the PEFA was added with other compounds that produce foam when added to water. In particular, certain amounts of a PEFA compound and another compound were added to water in a vial to determine the amount of foam height generated by the combination. A control using just sodium lauryl sulfate (SLS) was also evaluated to determine the height of foam from the use of that specific surfactant. Table 1 below illustrates the results below showing foam volume height in mL.

TABLE 1

| | 10 g | 10 g | | | | |
|---|---|---|---|---|---|---|
| | water + 0.5 g | water + 0.1 g | | 10 g | 10 g | |
| | PEFA + 0.05 g | SPAN 80 + | | water + 0.1 g | water + 0.1 g | 10 g water + |
| | SLS + 0.1 | 0.05 g SLS + | | PEFA + | Span20 + | 0.1 g SLS + |
| Time | gSpan20 | 0.5 g PEFA | 0.05 g SLS | 0.05 g SLS | 0.05 g SLS | 0.02 g PEFA |
| (mins) | (Example A) | (Example B) | (Example C) | (Example D) | (Example E) | (Example F) |
| 0 | 0 | 0 | 0 | O | 0 | 0 |
| 0.5 | 5 | 2.5 | 15 | 1 | 15 | 10 |
| 5 | 2.5 | 1.8 | 15 | 1 | 15 | 10 |
| 10 | 2.5 | 1.5 | 15 | 1 | 15 | 10 |
| 15 | 2.5 | 1.5 | 15 | 1 | 15 | 10 |
| 20 | 1.8 | 1 | 10 | 0.5 | 12 | 8 |

In each of the examples, the initial amount of foam formed was essentially zero. Over time as the foam formed, the foam height grew substantially for Examples C and E which included no PEFA compounds. The foam height of the sample illustrated by Example C (containing only SLS at 0.05 g) was significantly higher than the foam height of samples for Examples A, B, and D that had the same amounts of SLS, but also contained 0.1 g to 0.5 g of the PEFA compound. Thus, the PEFA compound in Examples, A, B, and D reduced or suppressed the foaming impact of SLS. Further, when evaluating the impact of the PEFA compound on foam generation, Example A showed about a 85% reduction in the amount of foam generated relative to the foam generated by Example E, which included the same components except for the PEFA compound.

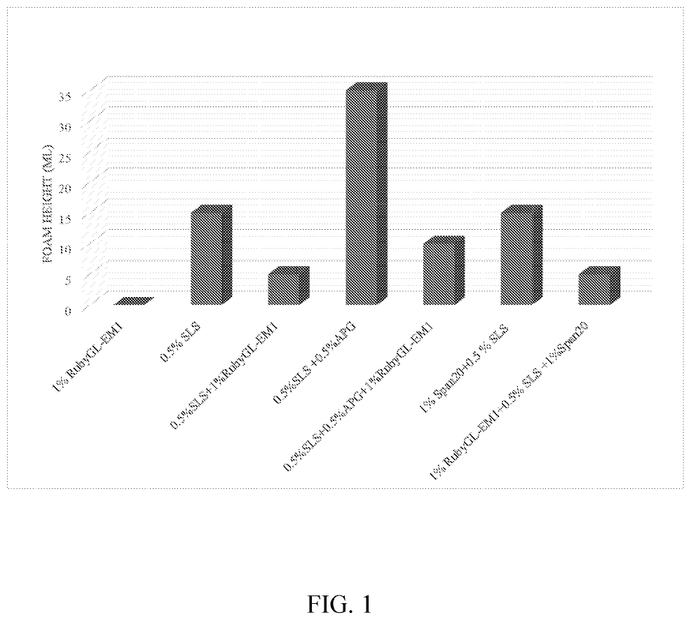

The impact of a PEFA compound on foam generation is further illustrated by the graph in FIG. 1. The PEFA sample illustrated by the first bar in FIG. 1 (referenced as 10% RubyGL-EM1) produced virtually zero foam when added to water. As expected, the second bar in FIG. 1 (referenced as 0.5% SLS) produced significant foaming due to the addition of SLS to water. The third bar in FIG. 1 (referenced as 0.5% SLS+1% RubyGL-EM1) illustrates that the addition of a PEFA compound to SLS reduced or suppressed the amount of foam generated by about 71%. The fourth bar in FIG. 1 (referenced as 0.5% SLS+0.5% APG) also generated significant foaming. APG is an alkyl polyglycoside surfactant. The fifth bar in FIG. 1 (referenced as 0.5% SLS+0.5% APG+1% RubyGL-EM1) further demonstrates that the addition of a PEFA compound to a composition including known foam-generating compounds will reduce the amount of foam generated. With respect to this fifth bar the amount of foam reduced or suppressed relative to the fourth bar was about 73%. The sixth bar in FIG. 1 (referenced as 1% SPAN20+0.5% SLS) also generated significant foaming. The seventh bar in FIG. 1 (referenced as 1% RubyGL-EM1+0.5% SLS+1% SPAN20) further demonstrates that the addition of a PEFA compound to a composition including known foam-generating compounds will reduce the amount of foam generated. With respect to this seventh bar the amount of foam reduced or suppressed relative to the sixth bar was about 71%.

Those skilled in the art will recognize or be able to ascertain using no more than routine experimentation, many equivalents to the specific embodiments in accordance with the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A foam control agent comprising one or more polyol lipids, wherein the one or more polyol lipids are selected from a polyol ester of fatty acids (PEFA) compound represented by Formula (I) or (II): 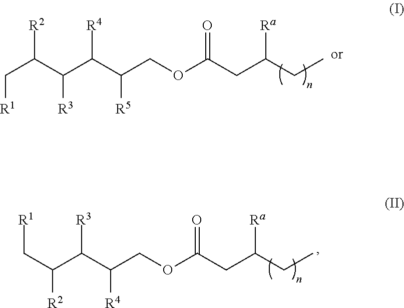
or salt or acid thereof,
wherein
R¹ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —COOH, —NO₂, —NH₂, —(CⱼHₖOₘ), —OH, —(O)C(O)R^{x}, —(O)R^{y}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R² is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R³ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁴ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R⁵ is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
R^{a} is selected from the group consisting of —H, —C(O)R^{x}, —R^{x}, —C(O)OR^{y}, —C(O)N(H)R^{y}, —(C₂H₅O)ₚR^{y}, —P(O)(OH)₂, —S(O₂)OH, —NO₂, —NH₂, —(CⱼHₖOₘ), —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, —(O)S(O₂)OH, —(O)NO₂, —(O)NH₂, and —(O)(CⱼHₖOₘ);
each R^{x} is independently C₁-C₃ alkyl, wherein R^{x} is optionally substituted with 1-7 instances of halogen;
each R^{y} is independently —H, or C₁-C₃ alkyl, wherein R^{x} is optionally substituted with 1-7 instances of halogen;
n is 2-20;
j is 2-12;
k is 4-26;
m is 1-16; and
each p is independently selected from 1-10.

2. A foam control agent comprising one or more polyol lipids, wherein the one or more polyol lipids are selected from
a) a PEFA compound of Formula (I) or (II) 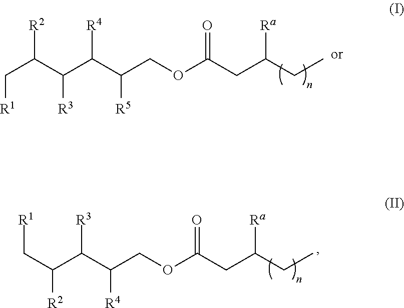
or salt or acid thereof,
wherein
R¹ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R² is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R³ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R⁴ is selected from the group consisting of —OH, —(O)C(O)R^{x}, —OR^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R⁵ is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
R^{a} is selected from the group consisting of —OH, —(O)C(O)R^{x}, —(O)R^{x}, —(O)C(O)OR^{y}, —(O)C(O)N(H)R^{y}, —(O)(C₂H₅O)ₚR^{y}, —(O)P(O)(OH)₂, and —(O)S(O₂)OH;
each R^{x} is independently C₁-C₃ alkyl, wherein R^{x} is optionally substituted with 1-7 instances of halogen;
each R^{y} is independently —H, C₁-C₃ alkyl, wherein R^{x} is optionally substituted with 1-7 instances of halogen;
n is 2-20;
j is 2-12;
k is 4-26;
m is 1-16; and
each p is independently selected from 1-10,
wherein
when n is 12 or 14 and R¹ is —OH or —(O)Ac, then R^{a} is not —(O)Ac or —(O)Me;
when n is 12 and R^{a} is —OH, then R² is not —OH or;
when n is 12 or 14, R^{a} is —OH, R² is OH, R³ is OH, and R⁴ is OH, then R¹ is (O)R^{x} or —(O) (C₂H₅O)ₚR^{y};
when n is 12 or 14, R¹ is —(O)S(O₂)OH, R² is —(O)S(O₂)OH, R³ is —(O)S(O₂)OH, R⁴ is —(O)S(O₂)OH, and R⁵ is —(O)S(O₂)OH, then R^{a} is —(O)C(O)R^{x}, R^{x}, —(O)C(O)OR^{x}, —(O)C(O)N(H)R^{x}, —(O)(C₂H₅O)ₚR^{y}, —(O)P(O)(OH)₂, or —(O)S(O₂)OH;
b) the group consisting of an acetylated C12:0-3-hydroxy fatty acid esterified to D-arabitol with 3 acetylations, an acetylated C14:0-3-hydroxy fatty acid esterified to D-mannitol with 4 acetylations, an acetylated C16:0-3-hydroxy fatty acid esterified to D-mannitol with 2 acetylations, an acetylated C16:0-3-hydroxy fatty acid esterified to D-arabitol with 2 acetylations, an acetylated C14:0-3-hydroxy fatty acid esterified to D-arabitol with 4 acetylations, an acetylated C14:0-3-hydroxy fatty acid esterified to D-mannitol with 5 acetylations, an acetylated C16:0-3-hydroxy fatty acid esterified to D-mannitol with 3 acetylations, an acetylated C16:0-3-hydroxy fatty acid esterified to D-arabitol with 3 acetylations, an acetylated C16:0-3-hydroxy fatty acid esterified to D-mannitol with 4 acetylations, an acetylated C18:0-3-hydroxy fatty acid esterified to D-mannitol with 2 acetylations, an acetylated C16:0-3-hydroxy fatty acid esterified to D-mannitol with 5 acetylations, an acetylated C16:0-3-hydroxy fatty acid esterified to D-mannitol with 4 acetylations, an acetylated C18:0-3-hydroxy fatty acid esterified to D-mannitol with 2 acetylations, an acetylated C16:0-3-hydroxy fatty acid esterified to D-mannitol with 5 acetylations, an acetylated C16:0-3-hydroxy fatty acid esterified to D-arabitol with 4 acetylations, an acetylated C18:0-3-hydroxy fatty acid esterified to D-mannitol with 3 acetylations, an acetylated C18:0-3-hydroxy fatty acid esterified to D-mannitol with 4 acetylations, an acetylated C18:0-3-hydroxy fatty acid esterified to D-arabitol with 3 acetylations, an acetylated C18:0-3-hydroxy fatty acid esterified to D-mannitol with 5 acetylations, an acetylated C18:0-3-hydroxy fatty acid esterified to D-arabitol with 4 acetylations, an acetylated C20:0-3-hydroxy fatty acid esterified to D-mannitol with 3 acetylations, an acetylated C20:0-3-hydroxy fatty acid esterified to D-mannitol with 4 acetylations; or
c) the group consisting of: 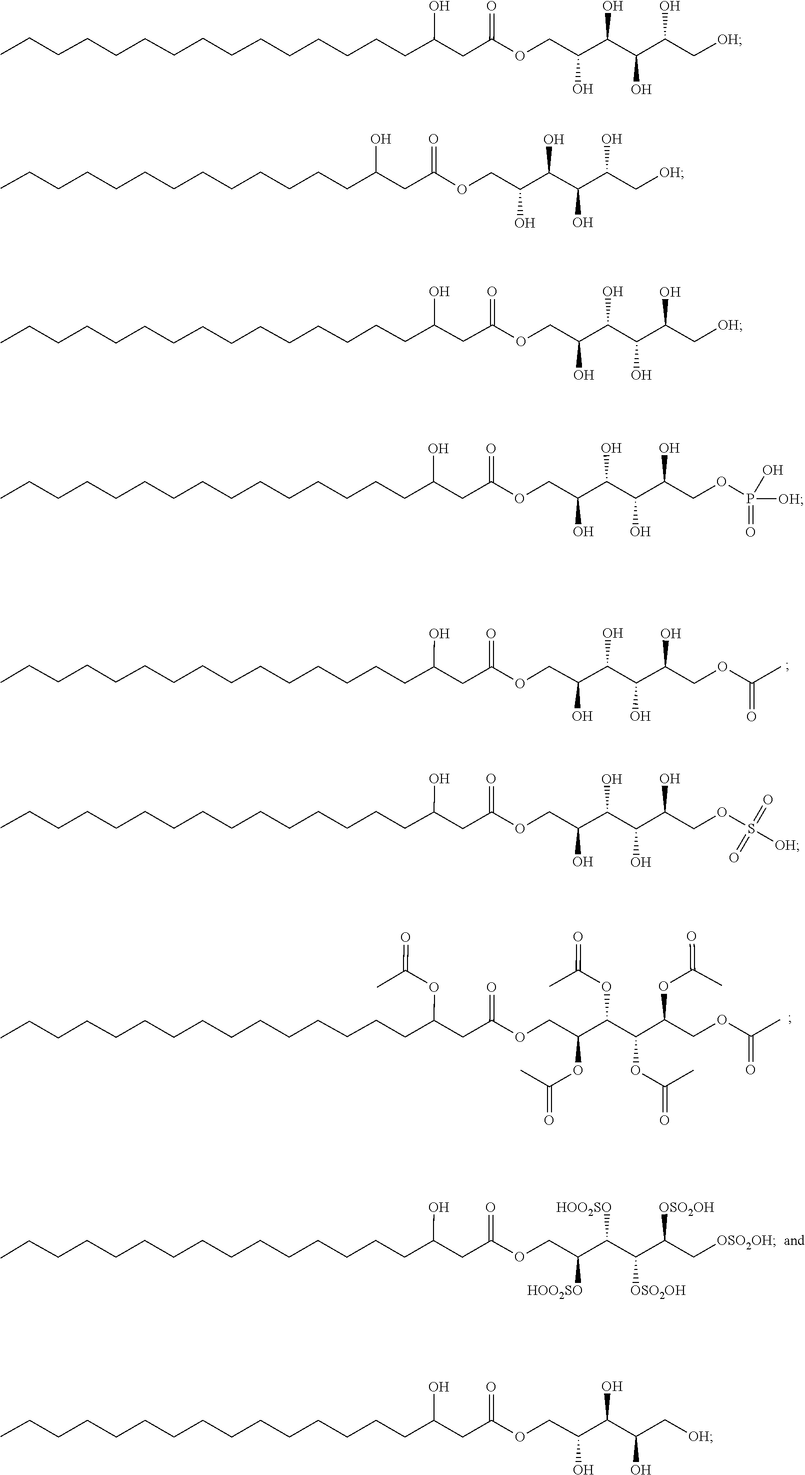
or salt or acid thereof.

3. The foam control agent of any of claims 1 or 2, wherein the foam control agent comprises two or more of the polyol lipids.

4. The foam control agent of claims 1-3, wherein the PEFA compound has an n value from 4 to 20.

5. The foam control agent of claims 1-3, wherein the one or more polyol lipids comprise pentitol polyol esters, hexitol polyol esters, or a combination thereof.

6. The foam control agent of claim 5, wherein the ratio of pentitol to hexitol polyol esters is about 5:1 or greater.

7. The foam control agent of claim 5, wherein the ratio of pentitol to hexitol polyol esters is about 20:1 or greater.

8. The foam control agent of claim 5, wherein the C16 and C18 fatty acid components of the pentitol and hexitol polyol esters can comprise about 20% to about 99% of the PEFA compound.

9. The foam control agent of claim 5, wherein the C16 and C18 fatty acid components of the pentitol and hexitol polyol esters can comprise about 30% to about 95% of the PEFA compound.

10. The foam control agent of claim 5, wherein the C16 and C18 fatty acid components of the pentitol and hexitol polyol esters can comprise about 40% or more of the PEFA compound.

11. The foam control agent of claims 1-10, wherein the PEFA compound is further represented by Formula (I-a) or (II-a): 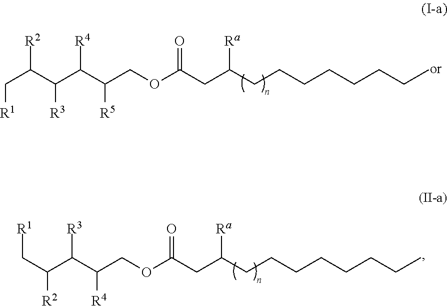
or salt or acid thereof,
wherein
n is 6-12.

12. The foam control agent of claims 1 or 2 comprising two or more polyol lipids.

13. A personal care composition comprising the foam control agent of claims 1 or 2.

14. The personal care composition of claim 13 is a cosmetic or dermatological composition.

15. The personal care composition of claim 13, wherein the foam control agent is a primary foam control agent.

16. The personal care composition of claim 15, further comprising a secondary foam control agent, wherein the secondary foam control agent is a non-PEFA foam control agent.

17. A foodstock composition comprising the foam control agent of claims 1 or 2.

18. The foodstock composition of claim 17, wherein the quantity of the foam control agent is 0.01% to 5%, by weight, of the composition.

19. The foodstock composition of claim 17, wherein the quantity of the foam control agent is 0.001% to 0.5%, by weight, of the composition.

20. The foodstock composition of claim 17, wherein the foam control agent is a primary foam control agent.

21. The foodstock composition of claim 20, further comprising a secondary foam control agent, wherein the secondary foam control agent is a non-PEFA foam control agent.

22. An agricultural composition comprising a foam control agent of claim 1 or 2.

23. The agricultural composition of claim 22 comprising two or more polyol lipids.

24. A paint composition comprising a foam control agent of claim 1 or 2.

25. The paint composition of claim 24 comprising two or more polyol lipids.

26. A coating composition comprising a foam control agent of claim 1 or 2.

27. The coating composition of claim 26 comprising two or more polyol lipids.

28. An adhesive composition comprising a foam control agent of claim 1 or 2.

29. The adhesive composition of claim 28 comprising two or more polyol lipids.

30. A sealant composition comprising a foam control agent of claim 1 or 2.

31. The sealant composition of claim 30 comprising two or more polyol lipids.

32. A foam control formulation comprising one or more of the foam control agents of claims 1 or 2.

33. The foam control formulation of claim 32, further comprising a solvent.

34. The foam control formulation of claim 32, wherein the foam control agent is a primary foam control agent.

35. The foam control formulation of claim 34, further comprising a secondary foam control agent, wherein the secondary foam control agent is a non-PEFA foam control agent.

36. A method of reducing or preventing the generation of foam, the method comprising adding the foam control agent of claims 1 or 2 before, during, or after the foam is generated.

37. The method of claim 36, wherein the foam is reduced by about 10% or more.

38. The method of claim 36, wherein the foam is reduced by about 50% or more.

39. The method of claim 36, wherein the foam is reduced by about 70% or more.
